(19) United States
(12) Patent Application Publication
(10) Pub. No.: US 2026/0248089 A1
(43) Pub. Date: Aug. 27, 2026

(54) PHYTOPHTHORA CAPSICI RESISTANT PEPPER

(71) Applicant: Enza Zaden Beheer B.V., Enkhuizen (NL)

(72) Inventors: Cornelis Walter Verweij, Enkhuizen (NL); Siyang Liu, Enkhuizen (NL); Petronella Martina Bleeker, Enkhuizen (NL); Robin Paul Huibers, Enkhuizen (NL)

(21) Appl. No.: 18/860,441

(22) PCT Filed: Apr. 26, 2023
(86) PCT No.: PCT/EP23/61031

§ 371 Date: Oct. 25, 2024

Related U.S. Application Data
(60) Provisional application No. 63/335,637, filed on Apr. 27, 2022.

(51) Int. Cl. A01H 1/00; A01H 5/08; A01H 6/82; C07K 14/415; C12Q 1/6895

(52) U.S. Cl. CPC A01H 1/1255; A01H 1/125; A01H 5/08; A01H 6/822; C07K 14/415; C12Q 1/6895; C12Q 2600/13

(57) ABSTRACT

The present invention relates to Phytophthora capsici (Pc) resistant Capsicum annuum (pepper) plants wherein said resistance is conferred by a combination of resistance genes and a resistance locus. The invention further relates to the combination of resistance genes and locus providing resistance to Pc and furthermore provides resistance to Xanthomonas campestris pv. vesicatoria (Xcv). The present invention further relates to methods for providing the plant of the C. annuum species being resistant to Pc and/or Xcv.

The present invention relates to Phytophthora capsici (Pc) resistant Capsicum annuum (pepper) plants, wherein said resistance is conferred by a combination of resistance genes and a resistance locus. The invention further relates to the combination of resistance genes and locus providing resistance to Pc and furthermore provides resistance to Xanthomonas campestris pv. vesicatoria (Xcv). The present invention further relates to methods for providing the plant of the C. annuum species being resistant to Pc and/or Xcv.

One of the significant threats to pepper production worldwide is Phytophthora blight. This disease is caused by the oomycete species Phytophthora capsici (Pc), which has a high degree of pathogen diversity. Phytophthora blight (also known as or Phytophthora rot) can infect all parts of the pepper plant, including the roots, stems, leaves, and fruit. The disease can cause wilting, stunting, and eventual death of the plant. In fruit, the disease can cause dark, sunken lesions that may lead to fruit rot. One of the challenges of managing Phytophthora blight in pepper plants is that the pathogen can survive in the soil for long periods of time, and can be spread by contaminated soil, water, tools, and equipment. The disease is also favored by wet, humid conditions, which can encourage the growth and spread of the pathogen.

Another significant threat to pepper production worldwide is Xanthomonas campestris pv. Vesicatoria (Xcv), which is a bacterial pathogen that causes bacterial spot disease in pepper plants. The bacteria can infect leaves, stems, and fruit of the plant, causing lesions that can lead to defoliation, reduced fruit yield, and poor fruit quality. Bacterial spot disease can spread rapidly through a field or greenhouse, especially under warm and humid conditions. Symptoms of bacterial spot disease in pepper plants typically include small, water-soaked lesions on leaves, stems, and fruit. As the disease progresses, the lesions can turn brown or black and become raised or sunken. The leaves may also become yellow and drop prematurely, and the fruit may develop lesions that can cause it to rot.

Management of both bacterial spot disease and Phytophthora blight in pepper plants involves a combination of cultural practices, such as removing and destroying infected plant material, rotating crops, and avoiding overhead irrigation, as well as the use of fungicides or bactericides. However, Resistant Xanthomonas and Phytophthora strains develop over time and are a recurring problem. It is important to note that prevention is key. A continued development of improved disease-resistant pepper varieties will help reduce the risk of infection and improve crop yields worldwide. Currently, control of this disease relies on extensive use of fungicides and therefore breeding for resistance is an important goal.

Considering the above, there is a need in the art to identify specific resistance genes for introgressing in a stepwise manner and breeding pepper varieties to provide a pepper plant having improved disease resistance, more specifically improved resistance against Phytophthora capsica (Pc) and/or Xanthomonas campestris pv. vesicatoria (Xcv).

It is an object of the present invention, amongst other objects, to address the above need in the art. The object of present invention, amongst other objects, is met by the present invention as outlined in the appended claims.

Specifically, the above object, amongst other objects, is met, according to a first aspect wherein a plant of the Capsicum annuum (C. annuum) species that is resistant to Phytophthora capsica (Pc), wherein the plant comprises a SAR8.2 resistance gene, that encodes for a SAR8.2 resistance protein, having at least 90%, preferably at least 95%, even more preferably 98%, most preferably 100% sequence identity with SEQ ID No.1, a Pc6.1 resistance locus, wherein the Pc6.1 resistance locus comprises a first marker comprising a T at position 54 of SEQ ID No.5 and/or a second marker comprising an A at position 101 of SEQ ID No.6, and a Xcv resistance gene, that encodes for a resistance Xcv protein having at least 90%, preferably at least 95%, even more preferably 98%, most preferably 100% sequence identity with SEQ ID No.4.

The Capsicum annuum pepper plant of the present invention comprises a SAR8.2 resistance gene on Chromosome 5 and a resistance locus on Chromosome 6, wherein the plant exhibits resistance to Phytophthora capsici. The resistance locus designated as Pc6.1 includes a first marker including a T at position 54 of SEQ ID No.5 and/or wherein Pc6.1 is linked to a first marker including a T at position 54 of SEQ ID No.5 and/or is linked to a second marker comprising an A at position 101 of SEQ ID No.6. SAR8.2 and Pc6.1 are both able to confer resistance to Phytophthora capsici (Pc) on its own. Importantly, the combination of the two provides an increased level of resistance to Pc compared to each separately. These two resistance sources are valuable additions to breeding new pepper varieties resistant to Pc and suitable for growth in specific regions. The plant of present invention further comprises an Xcv resistance gene. The Xcv gene is located on Chromosome 3 and provides the plant with resistance to Xanthomonas campestris pv. vesicatoria (Xcv). Therefore, the plant of present invention shows improved disease resistance to Pc and at the same time is resistant to Xcv.

According to another embodiment, the present invention relates to the plant, wherein the SAR8.2 resistance gene comprises a coding sequence having at least 90%, preferably at least 95%, even more preferably 98%, most preferably 100% sequence identity with SEQ ID No.1. In further embodiments of this aspect, which may be combined with any of the embodiments, a protein sequence encoded by the SAR8.2 resistance gene comprises SEQ ID No.2.

According to a preferred embodiment, the present invention relates to the plant, wherein the Xcv resistance gene comprises a coding sequence having at least 90%, preferably at least 95%, even more preferably 98%, most preferably 100% sequence identity with SEQ ID No.3.

According to yet another embodiment, the present invention relates to the plant, wherein the SAR8.2 resistance gene comprises at least one of the mutations selected from the group consisting of G48C, A179G and A14G as compared to a SAR8.2 gene encoding for a SAR8.2 protein (SEQ ID No.10) not providing resistance to Phytophthora capsici. The coding sequence of the SAR8.2 susceptible gene (i.e., the counterpart of the SAR8.2 resistance gene of the present disclosure, wherein the plant with the SAR8.2 (susceptible) gene exhibits susceptibility to Pc) includes SEQ ID No.9, and the protein sequence encoded by the SAR8.2 susceptible gene includes SEQ ID NO.10. SEQ ID NO: 9 includes an A at position 14, a G at position 48, and an A at position 179. SEQ ID No.10 includes an Asparagine (N) at residue 5, a Valine (V) at residue 17, and an Aspartic acid (D) at residue 60.

According to some embodiments, the SAR8.2 resistance gene encodes a SAR8.2 resistance protein that comprises a sequence that is at least 90% (e.g., at least 95%, at least 98%) identical to SEQ ID NO: 10 and comprises one or more mutations selected from N5S, V17L, and D60G with reference to a SEQ ID NO: 10. In some embodiments, the SAR8.2 resistance gene comprises at least one of the mutations selected from the group of G48C, A179G and A14G, as compared to a SAR8.2 gene (SEQ ID NO: 9) encoding for a SAR8.2 protein (SEQ ID No.10) that does not provide resistance to Phytophthora capsici.

According to a further embodiment, the present invention relates to the plant, wherein the Pc6.1 resistance locus is flanked by SEQ ID No.7 and SEQ ID No.8. Additional embodiments of this aspect, which may be combined with any of the embodiments.

According to another embodiment, the present invention relates to the plant, wherein the plant is furthermore resistant to Xanthomonas campestris pv. vesicatoria (Xcv).

According to a preferred embodiment, the present invention relates to the plant, wherein the plant is resistant to one or more of Xanthomonas campestris pv. vesicatoria (Xcv) races 3, 4, 5 and/or 6.

According to an additional embodiment, the present invention relates to the plant, wherein the SAR8.2 resistance gene is homozygous or heterozygous present in the genome of said plant, preferably homozygous.

According to yet another embodiment, the present invention relates to the plant wherein the Pc6.1 locus is homozygous or heterozygous present in the genome of said plant, preferably homozygous.

According to yet another embodiment, the present invention relates to the plant wherein the Xcv resistance gene is homozygous or heterozygous present in the genome of said plant, preferably homozygous.

According to yet another embodiment, the present invention relates to the plant wherein the plant is a sweet bell pepper.

According to a preferred embodiment, the present invention relates to the plant, wherein the SAR8.2 gene is obtainable from deposit accession number NCIMB 44129. Seeds are deposited at NCIMB Ltd, Ferguson Building, Craibstone Estate, Bucksburn, Aberdeen, AB21 9YA Scotland, on 9 Mar. 2023 under the number NCIMB 44129.

According to another preferred embodiment, the present invention relates to the plant, wherein the Pc 6.1 locus is obtainable from deposit accession number NCIMB 44129.

According to an additional embodiment, the present invention relates to the plant, wherein the Xcv locus is obtainable from deposit accession number NCIMB 44129.

The present invention, according to a further aspect, relates to a seed, tissue, plant cell, or plant part of the plant of any one of the preceding embodiments described in the first aspect of present invention, wherein the seed, tissue, or plant part comprises the SAR8.2 resistance gene, Pc6.1 resistance locus and Xcv resistance gene. Other plant parts include pepper cotyledons, hypocotyls, leaves, petioles, stems, flowers, pistils, ovaries, ovules, pericarps, embryo sacs, anthers, microspores, pollen grains, seeds, embryos, fruits, parts of fruits, roots, root tips, meristems, and the like. In another embodiment, the present disclosure is further directed to protoplasts, tissue culture, or cell culture produced from pepper plants of any of the above embodiments. In certain embodiments, tissue or cell culture of pepper plants of the present disclosure is produced from a plant part selected from root, root tip, meristematic cell, stem, hypocotyl, petiole, cotyledon, leaf, flower, anther, pollen, pistil, and fruit. In an additional embodiment, the present disclosure is further directed to pepper plants regenerated from the tissue or cell culture, where the plant has all of the morphological and physiological characteristics of pepper plants of any of the above embodiments.

The present invention, according to a further aspect, relates to a SAR8.2 resistance gene in combination with a Pc6.1 resistance locus and a Xcv resistance gene in a C. annuum plant for providing resistance to Phytophthora capsica, wherein the SAR8.2 resistance gene comprises a coding sequence having at least 90%, preferably at least 95%, even more preferably 98%, most preferably 100% sequence identity with SEQ ID No.1, the Pc6.1 resistance locus, preferably wherein the Pc6.1 resistance locus is flanked by SEQ ID No.7 and SEQ ID No.8, and the Xcv resistance gene comprises a coding sequence having at least 90%, preferably at least 95%, even more preferably 98%, most preferably 100% sequence identity with SEQ ID No.3. Preferably the SAR8.2 resistance gene, Pc6.1 resistance locus and Xcv resistance gene are homozygous present in plant, and preferably wherein said combination of SAR8.2 resistance gene, Xcv resistance gene and Pc6 resistance locus furthermore provides for a C. annuum plant that is resistant to Xanthomonas campestris pv. vesicatoria (Xcv). The SAR8.2 resistance gene encodes for a SAR8.2 resistance protein having at least 95% amino acid sequence identity, preferably 100% amino acid sequence identity with SEQ ID No.2. The Pc6.1 resistance locus comprises a first marker comprising a T at position 54 of SEQ ID No.5 and/or a second marker comprising an A at position 101 of SEQ ID No.6. The Xcv resistance gene encodes for a resistance Xcv protein having at least 95% amino acid sequence identity, preferably 100% amino acid sequence identity with SEQ ID No.4.

According to yet another embodiment, the present invention relates to a SAR8.2 resistance gene in combination with a Pc6.1 resistance locus and a Xcv resistance gene in a C. annuum plant for providing resistance to Phytophthora capsica, wherein the SAR8.2 resistance gene in combination with a Pc6.1 resistance locus and a Xcv resistance gene in a C. annuum plant according to claim 15, wherein the SAR8.2, Pc6.1 and Xcv resistance genes are homozygous present in said C. annuum plant.

According to a further embodiment, the present invention relates to a SAR8.2 resistance gene in combination with a Pc6.1 resistance locus and a Xcv resistance gene in a C. annuum plant for providing resistance to Phytophthora capsica, wherein the SAR8.2 resistance gene in combination with a Pc6.1 resistance locus and a Xcv resistance gene in a C. annuum plant according to claim 15 or 16, wherein said combination furthermore provides for resistance to Xanthomonas campestris pv. vesicatoria (Xcv).

The present invention, according to a further aspect, relates to a DNA construct comprising a SAR8.2 resistance gene in combination with a Pc6.1 resistance locus and a Xcv resistance gene of any one of the embodiments described herein, wherein one or more of said genes and/or said locus is operably linked to a promoter.

The present invention, according to yet a further aspect, relates to a method for providing a plant of the C. annuum species according to the preceding embodiments described herein, wherein the method comprises selecting a pepper plant that is resistant to Phytophthora capsici, wherein said selection comprises establishing the presence of one or more resistance genes or locus selected from the group consisting of a SAR8.2 resistance gene, a Pc6.1 resistance locus and a Xcv resistance gene, wherein
the SAR8.2 resistance gene comprises a coding sequence having at least 90%, preferably at least 95%, more preferably at least 98% sequence identity with SEQ ID No.1,
the Pc6.1 resistance locus comprises a first marker comprising a T at position 54 of SEQ ID No.5 and/or a second marker comprising an A at position 101 of SEQ ID No.6, and;
the Xcv resistance gene comprises a coding sequence having at least 90%, preferably at least 95%, more preferably at least 98% sequence identity with SEQ ID No.3.

The present invention, according to yet a further aspect, relates to a method for providing a plant of the C. annuum species according to the preceding embodiments described herein, wherein the method comprises the steps of;
a) selecting a pepper plant that is resistant to Phytophthora capsici, wherein said selection comprises establishing the presence of one or more resistance genes or locus selected from the group consisting of a SAR8.2 resistance gene, a Pc6.1 resistance locus and a Xcv resistance gene, wherein
   the SAR8.2 resistance gene comprises a coding sequence having at least 90%, preferably at least 95%, more preferably at least 98% sequence identity with SEQ ID No.1,
   the Pc6.1 resistance locus comprises a first marker comprising a T at position 54 of SEQ ID No.5 and/or a second marker comprising an A at position 101 of SEQ ID No.6, and;
   the Xcv resistance gene comprises a coding sequence having at least 90%, preferably at least 95%, more preferably at least 98% sequence identity with SEQ ID No.3, and
b) transferring, for example by crossing, the identified one or more resistance genes of step a) into a C. annuum plant thereby conferring Phytophthora capsici resistance to said C. annuum plant; and
c) optionally, perform steps a and b until the obtained C. annuum comprises the SAR8.2 resistance gene, the Xcv resistance gene and the Pc6.1 resistance locus and in its genome.

According to a following embodiment, the present invention relates to the method, wherein after step b) a first C. annuum plant is selected that is resistant to Phytophthora capsici and is crossed with a second C. annuum plant that is not resistant to Phytophthora capsici, and subsequently selecting a C. annuum plant that is resistant to Phytophthora capsici.

According to a further embodiment, the present invention relates to the method, wherein in step a) establishing the presence of the SAR8.2 resistance gene in C. annuum plant is performed by a marker comprising SEQ ID No: 1.

According to another embodiment, the present invention relates to the method, wherein the presence of the Pc6.1 resistance locus in C. annuum plant is established by a marker comprising a T at position 54 of SEQ ID NO:5 and/or a marker comprising an A at position 101 of SEQ ID NO: 6 and/or wherein the resistance locus is flanked by a marker comprising SEQ ID NO: 7 and/or a marker comprising SEQ ID NO: 8.

According to yet another embodiment, the present invention relates to the method, wherein the presence of the Xcv resistance gene in C. annuum plant is established by a marker comprising SEQ ID No.3.

According to a next embodiment, the present invention relates to the method, wherein the plant of the C. annuum species provided, is resistant to Phytophthora capsica and Xanthomonas campestris pv. Vesicatoria.

The present invention, according to a further aspect, relates to the use of a marker for establishing the presence of a SAR8.2, and/or Xcv resistance gene(s) and/or Pc6.1 resistance locus according to the preceding embodiments described in the proceeding aspects of present invention of the pepper plant, wherein
for SAR8.2 resistance gene the marker comprises SEQ ID No.1,
for Pc6.1 resistance locus the marker is one or more selected from the group consisting of SEQ ID No.5, SEQ ID No.6, and SEQ ID No.7 and SEQ ID No.8,
for Xcv resistance gene the marker comprises SEQ ID No.3.

The present invention will be further detailed in the following examples and figures wherein:

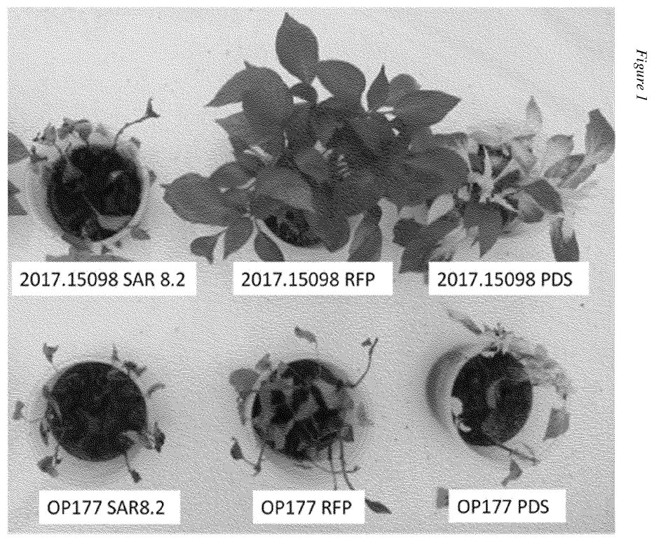

FIG. 1 shows a representative image of the Phytophthora capsici (Pc) resistance phenotype of ‘2017.15098’ plants (resistant background; top row) and ‘OP177’ plants (susceptible background; bottom row) with SAR8.2, RFP, or PDS genes (from left to right in each row) silenced by TVR-VIGS.

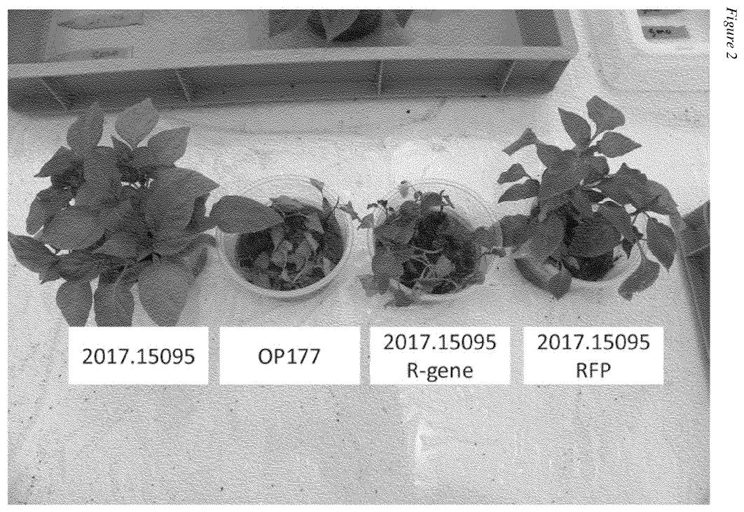

FIG. 2 shows a representative image of the Phytophthora capsici (Pc) resistance phenotype of a ‘2017.15095’ plant (resistant background; left), an ‘OP177’ plant (susceptible background; second from left), a ‘2017.15095’ plant with Pc6.1 silenced by TVR-VIGS (“2017.15095 R-gene”; second from right), and a ‘2017.15095’ plant with the RFP gene silenced by TVR-VIGS (“2017.15095 RFP”; right).

### EXAMPLES

### Identification of a Resistance Locus to Phytophthora capsici (Pc) on Capsicum annuum (Pepper) Chromosome 5

The following example describes the production of a resistance mapping population in pepper to identify the genetic basis of Pc resistance. The example further describes resistance phenotyping and fine mapping of the population, as well as candidate gene testing using Virus Induced Gene Silencing (VIGS).

The hot pepper isolate ‘FLORIDA ELITE’ is used as the original resistance donor source for the screening. ‘Maor’, a sweet bell pepper variety, is used as the original susceptible source for the screening. ‘CAPS6434’ is an introgression line produced from the cross of ‘FLORIDA ELITE’×‘Maor’ that contains a long Chr05 introgression from ‘FLORIDA ELITE’, and is resistant to Pc. ‘CAPS6436’ is an introgression line produced from the cross of ‘FLORIDA ELITE’×‘Maor’ that contained a shorter Chr05 introgression from ‘FLORIDA ELITE’, and is susceptible to Pc. Both ‘CAPS6434’ and ‘CAPS6436’ harbor only a single chromosomal introgression derived from ‘FLORIDA ELITE’ (located on Chr05), and the chromosomal introgression of ‘CAPS6436’ is slightly smaller compared to ‘CAPS6434’.

10 seeds for each genotype and gene were planted in plastic beakers on rockwool plugs. For each genotype and gene, 3 replicates were tested against a Pc isolate. When plants were at the 4-5 leaf stage, 15,000 spores of Phytophthora capsici (in the examples Pc isolate Q108 was used) were inoculated into each beaker. Plant growth and phenotype were analyzed on days 13 and 17 after Pc inoculation. Pc resistance phenotypes for the individual plants were either scored as resistant (R) or susceptible(S). As 3×10 (total of 30) plants per line were used in each experiment, the level of resistance per line was calculated as a percentage to produce a 0-100 score. In a resistant phenotype the roots and shoots grew normally and no lesion was observed; in a susceptible phenotype, root or shoot growth was stopped, and plant wilting and/or girdling was observed.

Phenotyping with Pc showed that ‘CAPS6434’ was 87% resistant to Pc and that ‘CAPS6436’ was 0% resistant to Pc (data not shown). In other words, 87% of plants showed no wilting (CAPS6434) versus all plants showed wilting (CAPS6436). It is thought that the differences in the introgression were accountable for the differences in the resistance patterns. Offspring with an ~3 million bp region from ‘FLORIDA ELITE’ showed resistance to Pc. This result meant that the ‘CAPS6434’ and ‘CAPS6436’ lines were suitable for use in the fine mapping. Table 1, below, shows the Pc resistance phenotype scoring at 17 days post infection listing the recombinants of the F2 and F3 families and not the CAPS6434.

Table 1, below, shows the recombinant screening data and the Pc resistance score. Based on the recombinant screening, the genetic region conferring resistance was between 22.751.611 and 32.345.327 on Zunla-1 v1.0 reference genome (Table 1).

TABLE 1
Physical position on FLORIDA ELITE v1.55 reference genome and Pc resistance score.

| | Physical position on FLORIDA ELITE V1.55 reference genome | | | | | | | |
|---|---|---|---|---|---|---|---|---|
| | | | | | | | | Pc |
| Recombinant | | | | | | | | resistance |
| family name | 22.751.611 | 28.041.969 | 27.986.976 | 28.878.718 | 30.101.411 | 32.345.327 | Chr06 | score |
| SLI-352 | P | P | P | P | P | A | A | 91 |
| SLI-361 | P | P | P | P | P | P | A | 68 |
| SLI-371 | P | P | P | P | P | P | A | 63 |
| SLI-377 | A | A | A | A | A | P | A | 0 |
| SLI-390 | A | A | A | A | A | P | A | 0 |
| SLI-458 | A | A | A | A | A | A | A | 0 |
| SLI-437 | P | P | P | P | P | A | A | 20 |

A stand for ‘absence’ of the resistance conferring genomic region, ‘P’ for ‘present’.

The susceptible ‘CAPS6436’ was 0% resistant to Pc likely due to the slightly smaller single chromosomal segment on Chr05 compared to ‘CAPS6434’. The larger chromosome of ‘CAPS6434’ is used to develop a mapping population.

### Gene Validation Using VIGS

We examined the region providing Pc resistance and tried to identify if one or more of these transcripts indeed carried resistance to Pc. Therefore, a Virus Induced Gene Silencing (VIGS) analysis is performed. Tobacco rattle virus (TRV)-derived VIGS vectors have been abundantly described to study gene function in plants such as Arabidopsis thaliana, Nicotiana benthamiana, Solanum lycopersicum and other plants (see for example Huang C, Qian Y, Li Z, Zhou X.: Virus-induced gene silencing and its application in plant functional genomics. Sci China Life Sci. 2012; 55 (2): 99-108).

As such, three VIGS constructs were developed VIGS-1 (SAR8.2) Table 2, VIGS-2 (PDS) and VIGS-3 (RFP), wherein each construct VIGS-1, 2, or 3 specifically targets transcript 1, 2, or transcript 3, respectively.

TABLE 2
VIGS-1 construct SAR8.2

| VIGS construct | Sequence |
|---|---|
| VIGS-1 (SEQ | ATGGTTTCCAAAAGTAGTATTTTTATTTGCCTT |
| ID No. 11) | TCTTTGATTATTCTAGTGATAATGTCCACACAG |
| SAR8.2 | ATTGTTGCCAGGGAGATGACTTCTGAGGCATCT |
| | GCTTCGCTGACCCAAGCGATGAATGGAAATAAC |
| | ATTAGTGAGACTAAGAAAGTTGGACGCCATTTA |
| | GTGAAAGGTCTTGATAAAATCTTCAAAGCTGGC |
| | AAAGTTATATATTGTAAAACTTGCAAAACTTGT |
| | CATGGCCGTTGTGACTATTGTTGTGCCTAA |

An Agrobacterium tumefaciens strain harboring the TRV containing sequence targeting SAR8.2 (SEQ ID No.11), phytoene desaturase (PDS), or red fluorescent protein (RFP) genes are each inoculated into 3 ml LC/LB media containing the appropriate antibiotics (Carbenicillin, Gentamicin, Kanamycin 1 ul/ml, and Rifampicin 0.5 ul/ml). After incubation at 28° C., 200 rpm for 18 hours, 100 μl of each culture was added to 250 ml sterilized flasks containing 22.5 mL of LC/LB, 2.5 μl 100 mM MES buffer (adjust pH to 5.8 with KOH), 25 μL 40 mg/mL ACS dissolved in DMSO, and Carbenicillin, Gentamicin, Kanamycin 1 μl/ml, and Rifampicin 0.5 μl/ml. After incubation at 28° C. at 200 rpm for 18 hours, the separate A. tumefaciens strains were collected by centrifuging at 4000 rpm for 10 minutes. The A. tumefaciens pellets are resuspended in about 5 ml MMA buffer (10 mM MES buffer, 10 mM MgCl2, 200 μM ACS dissolved in DMSO), then incubated at 28° C. at 200 rpm for 1-3 hours. After incubation, the OD of each A. tumefaciens suspension is adjusted to 1.0, and the A. tumefaciens strain harboring the TRV1 vector and the A. tumefaciens strain harboring the TRV2 vector containing sequence targeting SAR8.2, PDS, or RFP genes are mixed in a 1:1 ratio. The mixtures are incubated at room temperature in a greenhouse for 1-2 hours, then infiltrated into both cotyledons of each plant using a syringe (without needle), until the entire cotyledons are fully infiltrated with the A. tumefaciens mixtures. The PDS silencing phenotype is visually assayed by bleaching of leaves, and is visible approximately 3 weeks after A. tumefaciens infiltration, see Table 3. RFP is included as a control to show the effect of the VIGS procedure without altering the expression of a plant gene. RFP is transiently expressed in the plant cells after A. tumefaciens infection.

10 seeds for each genotype and gene are planted in plastic beakers on rockwool plugs. On average, each beaker contained 8 plants after germination. For each genotype and gene, 3 replicates are tested for resistance against a Pc isolate. In this example, 15,000 Pc spores are inoculated into each beaker 23 days after infiltration of the A. tumefaciens for the VIGS silencing experiment. The second inoculation is done 3 days after the first. Plant growth and phenotype are analyzed on day 17 after Pc inoculation. Pc resistance phenotypes were rated either as resistant or susceptible, conform described above.

FIG. 1 and Table 3 show the phenotyping results after TRV VIGS silencing and Pc inoculation at various time point after inoculation. Pc resistance phenotypes were scored on a scale of 1-100, with 100% being completely resistant.

As shown in FIG. 1 and Table 3, silencing of SAR8.2 with VIGS resulted in susceptibility in a Pc resistant background. Controls infiltrated with VIGS constructs but not inoculated with Pc are all scored as 100, having a resistant phenotype (data not shown). Line ‘2017.15098’ is the resistant background (carrying only SAR8.2 gene at Chromosome 5) and OP177 was the susceptible background (see also Table 6).

TABLE 3
Phenotyping results after TRV VIGS
silencing and inoculation with Pc.

| | VIGS | |
|---|---|---|
| | construct | Resistance score at |
| Pepper line | target | day 17 |
| 2017.15098 | SAR8.2 | 1 |
| 2017.15098 | RFP | 100 |
| 2017.15098 | PDS | 97 |
| OP177 | SAR8.2 | 0 |

After analyzing the genomic region between markers 22.751.611 and 32.345.327 on the FLORIDA ELITE v1.55 reference genome and the genes present in this region, gene SAR8.2 positioned on 25.301.418, in the QTL region on Chromosome 5 is selected as the candidate gene for further study. The TRV VIGS approach described above is used to reduce the expression of SAR8.2 to elucidate whether this gene is indeed involved in Pc resistance. Pepper line ‘2017.15098’, which is homozygous for the SAR8.2 gene, and pepper line ‘OP177’, which is lacking the SAR8.2 gene, were used in this study. Phytoene desaturase (PDS) gene (showing bleaching if PDS expression is reduced) and red fluorescent protein (RFP) gene were used as controls for the VIGS assay.

Based on publicly available reference genomes, SNPs are identified between the ‘FLORIDA ELITE’ SAR8.2 complete coding sequence (CDS, SEQ ID No.1) and the ‘Maor’ complete CDS (SEQ ID No.9). SNP 1: adenine (A) to guanine (G) mutation at position 14 compared to ‘Maor’ SAR8.2 CDS (SEQ ID No.9), which corresponds to position Chr5: 25.300.613 of the FLORIDA ELITE v1.55 reference genome; SNP 2: G to cytosine (C) mutation at position 48 compared to ‘Maor’ SAR8.2 CDS (SEQ ID No.9), which corresponds to position Chr5:25.300.647 of the FLORIDA ELITE v1.55 reference genome; and SNP 3: A to G mutation at position 179 compared to ‘Maor’ SAR8.2 CDS (SEQ ID No.9), which corresponds to position Chr5:25.301.483 of the FLORIDA ELITE v1.55 reference genome.

The pepper lines containing the ‘FLORIDA ELITE’ SAR8.2 are resistant to Pc, indicating that the SAR8.2 gene in ‘FLORIDA ELITE’ provides Pc resistance. Moreover, SAR8.2 is shown to be the relevant gene for Pc resistance using a VIGS silencing approach. VIGS silencing of the SAR8.2 gene results in susceptibility to Pc.

### Example 2: Identification of a Resistance Locus to Pc on Pepper Chromosome 6

In Example 2 the candidate Pc resistance locus is mapped on Chromosome 6. The hot pepper isolate ‘Florida Elite is used as the original resistance donor source for the screening. The pepper variety ‘OP177’ is used as the original susceptible source for the screening.

‘FLORIDA ELITE’ and ‘OP177’ are crossed to produce an F1 population. The F1 population is selfed to produce a segregating F2 population. Individuals in the F2 population are mapped and screened for recombinants. Those identified are selfed to produce a F3 population, which is also mapped and screened for recombinants and again selfed to produce an F4 population. Table 5 shows the recombinant screening data and the Pc resistance phenotyping score of a Pc isolate of the F4 population, wherein ‘A’ stands for ‘absence’ and ‘P for ‘presence’ of the resistance conferring genomic region.

The results obtained suggest that the region conferring resistance to Pc is between position 215.787.906 and 216.438.623 on the Zunla-1 v1.0 reference genome. The region identified as conferring resistance is a larger region comprising multiple genes.

TABLE 5
Physical position on Zunla-1 v1.0 reference genome and the Pc resistance phenotyping score.

| F4 | | | | | | | | | |
|---|---|---|---|---|---|---|---|---|---|
| recombinant | Position on Zunla-1 (v1.0) reference genome | | | | | | | | Pc |
| genotype | 208,668,344 | 213,852,743 | 214,349,257 | 215,787,906 | 216,077,906 | 216,438,623 | 216,775,624 | 218,345,687 | score |
| 2017.15101 | A | P | P | P | P | P | P | P | 75 |
| 2017.16359 | A | P | P | P | P | P | P | P | 100 |
| 2017.16363 | A | A | P | P | P | P | P | P | 92 |
| 2017.16370 | A | A | P | P | P | P | P | P | 85 |
| 2017.15118 | A | A | A | P | P | P | P | P | 70 |
| 2017.16372 | A | A | A | P | P | P | P | P | 94 |
| 2017.15122 | A | A | A | A | P | P | P | P | 61 |
| 2017.15129 | A | A | A | A | A | A | P | P | 13 |
| 2017.16380 | A | A | A | A | A | A | P | P | 0 |
| 2017.16387 | P | P | P | P | P | P | A | A | 100 |
| 2017.15153 | P | P | P | P | P | P | A | A | 85 |
| 2017.16404 | P | P | P | P | P | A | A | A | 91 |
| 2017.16409 | P | P | P | P | P | A | A | A | 50 |
| 2017.16411 | P | P | P | P | A | A | A | A | 0 |
| 2017.16414 | P | P | P | P | A | A | A | A | 2 |
| 2017.15177 | P | P | A | A | A | A | A | A | 32 |
| 2017.15181 | P | P | A | A | A | A | A | A | 24 |
| 2017.15183 | P | A | A | A | A | A | A | A | 1 |
| 2017.15188 | P | A | A | A | A | A | A | A | 2 |

### VIGS Silencing of Candidate Pc Resistance Gene on Chromosome 6

Pc6.1 is selected for testing with the TRV VIGS approach as described in Example 1. TRV VIGS are now used to reduce the expression of Pc6.1 to elucidate whether Pc6.1 is indeed involved in Pc resistance.

TABLE 6
VIGS-4 construct Pc6.2 resistance locus

| VIGS construct | Sequence |
|---|---|
| VIGS-4 (SEQ | GGAGGGAACGAGTTTCAGGTCCTTGGCCAGAAT |
| ID No. | AATATCCCATTATTCAAGTAGCTCTTAAGCACC |
| 12) Pc6.28 | ACCAGAACAGGTTCTTCTCTATTCTTTTGTTTC |
| | TTAAGCTTTAAGGTAATTAGGAAAATCAACCCA |
| | GCTTTTGTGTGTGTAAATTTACATAGTGGGATA |
| | GCTCTGAGAGTTTGCTGCCACCGAACGCTGGCA |
| | ATTTCAACTGTAAGTTTCTCTTTGAAACATACA |
| | TGGAGGTTCCTATTGTCTGCTATTGGTAGCCTC |
| | TCTACTATATGAAAGTTATCCTATATCTATGTT |
| | TTG |

Pc resistant pepper variety ‘FLORIDA ELITE’ and Pc susceptible pepper variety ‘OP177’ are used in this VIGS study. Additionally, two F3 lines produced are also included. One of the F3 lines (‘2017.15095’) contains Pc6.1 in a homozygous state but does not have the resistance gene of SAR8.2 (Table 7). The other F3 line (‘2017.15094’) contains both Pc6.1 resistant locus and the resistance gene of the SAR8.2 gene in homozygous state (Table 6). Furthermore, independent of resistance gene silencing the PDS gene is silenced as well that serves as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly. Next to the PDS gene (showing bleaching if PDS expression is reduced), the red fluorescent protein (RFP) gene was used as control for the VIGS assay.

FIG. 2 and Table 7 (below) show the phenotyping results after TRV VIGS silencing of Pc6.1 and Pc inoculation.

TABLE 7
Presence of the SAR8.2 resistance gene and Pc6.1 resistance
locus in pepper lines tested in VIGS assays.

| Pepper Lines | SAR8.2 on Chr05 | Pc6.1 on Chr06 |
|---|---|---|
| ‘2017.15098’ | + | − |
| ‘2017.15095’ | − | + |
| ‘2017.15094’ | + | + |
| ‘OP177’ | − | − |

‘+’ homozygous presence of said gene/locus
‘−’ absence of said gene/locus

The numerical results shown in Table 8 are percentages. Individual plants are scored according to their Pc resistance phenotype (i.e., as resistant (R) or susceptible(S). In a resistant phenotype, the roots and shoots grow normally and no lesion was observed; in a susceptible phenotype, root or shoot growth is stopped, and plant wilting and/or girdling is observed, as depicted in FIG. 1. As multiple replicates are tested (usually 30 plants divided over 3× pots with 10 plants each), the final score in Table 8 is shown as the percentage of all plants showing the R phenotype.

Accordingly, a score of 100 represents complete resistance (30 out of 30 scored ‘R’), while a score of 0 represents complete susceptibility. The TRV VIGS silencing data shows that silencing the putative Pc6.1 gene on Chromosome 6 resulted in increased susceptibility in a Pc resistant background (Figure. 2 and Table 8), although plants still show a level of Pc resistance.

TABLE 8
Phenotyping results after TRV VIGS silencing and inoculation with Pc.

| | VIGS construct | Number of days after Pc inoculation (dpi) | | | |
|---|---|---|---|---|---|
| Pepper line | target | 1 day | 9 days | 13 days | 16 days |
| ‘2017.15095’ | Pc6.1 | 100 | 77 | 13 | 2 |
| ‘2017.15095’ | PDS | 100 | 82 | 64 | 38 |
| ‘2017.15095’ | RFP | 100 | 74 | 69 | 51 |
| ‘OP177’ | Pc6.1 | 100 | 62 | 12 | 0 |
| ‘OP177’ | PDS | 100 | 64 | 7 | 0 |
| ‘OP177’ | RFP | 100 | 62 | 12 | 2 |
| ‘2017.15094’ | Pc6.1 | 100 | 82 | 72 | 36 |
| ‘2017.15094’ | PDS | 100 | 86 | 80 | 56 |
| ‘2017.15094’ | RFP | 100 | 73 | 88 | 79 |
| ‘OP177’ | No VIGS control | 100 | 63 | 1 | 0 |
| ‘2017.15094’ | No VIGS control | 100 | 100 | 100 | 100 |
| ‘2017.15095’ | No VIGS control | 100 | 100 | 100 | 97 |
| ‘OP177’ | No Pc control | 100 | 100 | 100 | 100 |
| ‘2017.15094’ | No Pc control | 100 | 100 | 100 | 100 |
| ‘2017.15095’ | No Pc control | 100 | 100 | 100 | 100 |

Pc6.1 resistance locus falls outside the region flanked by SNPmarker_1 and SNPmarker_2 (see Example 1) when respective DNA sequences are BLASTed against the FLORIDA ELITE V1.55 reference genome (Table 9). The Pc6.1 resistance locus comprises a first marker comprising a T at position 54 of SEQ ID No.5 and/or a second marker comprising an A at position 101 of SEQ ID No.6). When Pc6.1 resistance locus is mapped in Zunla-1 v1.0, which is not annotated in FLORIDA ELITE v1.6. Pc6.1 locus is flanked by SNPmarker_1 and SNPmarker_2 when these sequences are BLASTed against the Zunla-1 v1.0 reference genome (Table 9).

TABLE 9
Putative Pc6.1 gene mapping relative to SNP markers.

| | Reference | | | |
|---|---|---|---|---|
| Query ID | Genome | Subject ID | Subject Start | Subject End |
| SNPmarker_1 | Zunla-1 v1.0 | Chr06 | 216.422.011 | 216.420.692 |
| (SEQ ID No. 7) | FLORIDA ELITE | Pepper.v.1.55.chr06 | 3.476.545 | 3.475.226 |
| | V1.55 | | | |
| | FLORIDA ELITE | chr06 | 3.589.508 | 3.588.189 |
| | V1.6 | | | |
| SNPmarker_2 | Zunla-1 v1.0 | Chr06 | 215.792.352 | 215.792.052 |
| (SEQ ID No. 8) | FLORIDA ELITE | Pepper.v.1.55.chr06 | 4.174.618 | 4.174.918 |
| | V1.55 | | | |
| | FLORIDA ELITE | chr06 | 4.287.581 | 4.287.881 |
| | V1.6 | | | |
| 1^{st} marker - Pc6.1 | Zunla-1 v1.0 | Chr06 | 216.230.359 | 216.230.859 |
| (SEQ ID No. 5) | FLORIDA ELITE | Pepper.v.1.55.chr06 | 3.291.241 | 3.291.745 |
| | V1.55 | | | |
| | FLORIDA ELITE | chr06 | 3.404.204 | 3.404.534 |
| | V1.6 | | | |
| 2^{nd} marker - Pc6.1 | Zunla-1 v1.0 | Chr06 | 216.167.689 | 216.168.189 |
| (SEQ ID No. 6) | FLORIDA ELITE | Pepper.v.1.55.chr06 | 3.287.263 | 3.287.514 |
| | V1.55 | | | |
| | FLORIDA ELITE | chr06 | NA | NA |
| | V1.6 | | | |
| Pc6.1 on | Zunla-1 v1.0 | Chr06 | 216.169.401 | 216.169.600 |
| Chromosome 6 | FLORIDA ELITE | Pepper.v.1.55.chr06 | NA | NA |
| | V1.55 | | | |
| | FLORIDA ELITE | chr06 | 3.401.401 | 3.401.675 |
| | V1.6 | | | |

Pepper lines containing the region conferring resistance on Chromosome 6 are resistant to Pc. VIGS silencing of the candidate resistance locus Pc6.1 on Chromosome 6 results in susceptibility to Pc.

The resistance gene of the SAR8.2 gene on Chromosome 5 and Pc6.1 resistance locus on Chromosome 6 likely both function separately, and have an additive and more likely a synergistic effect when simultaneously present to confer improved resistance to Pc.

### Example 3: Phytophthora capsici Resistance of Pepper Lines with Different Chr05 and Chr06 Genetics

The following example describes the testing of pepper lines with different Chr05 and Chr06 genetics.

The pepper lines provided in Table 10 are tested for Pc resistance as described in Examples 1 and 2. The resistance phenotype is assayed 13 days after inoculation. Table 10, below, provides the tested pepper lines and the results of Pc resistance phenotyping. The columns “Chr05” and “Chr06” indicate whether a resistant gene of Sar8.2 or the resistance locus on Chr06 is present (R) or absent(S).

The column “Pc resistance score” provides the results of Pc resistance phenotyping in percentages, as described in Example 1 and 2 (100=all resistant; 0=none resistant).

TABLE 10
Chr05 and Chr06 genetics and Pc resistance phenotyping results.

| Pepper line | Chr05 | Chr06 | Pc resistance score |
|---|---|---|---|
| 2017.15092 | R | S | 65 |
| 2017.15093 | R | R | 100 |
| 2017.15094 | R | R | 95 |
| 2017.15095 | S | R | 70 |
| 2017.15096 | R | R | 100 |
| 2017.15097 | R | R | 95 |
| 2017.15098 | R | S | 75 |
| 2017.15099 | R | S | 65 |
| OP177 | S | S | 0 |
| Florida Elite | R | S | 100 |

As can be seen from Table 10, each of the resistance genes of Sar8.2 on Chr05 and the resistance locus on Chr06 is able to provide a level of resistance individually. However, the resistance gene of Sar8.2 on Chr05 together with the resistance locus on Chr06 are both needed for complete resistance (100%) to Pc. This can be seen, for example, in lines ‘2017.15093’ and ‘2017.15096’.

### Example 4: Pepper Plant Resistant to Phytophthora capsici and Xanthomonas campestris pv. vesicatoria (Xcv)

In this example an embodiment of present invention comprising resistance genes for Pc and Xcv is tested for both Pc and Xcv. The embodiment pepper line ‘E20B.30329’ comprises the SAR8.2 resistance gene (SEQ ID No.1), the Pc6.1 resistance locus on Chromosome 6, and the Xcv resistance gene (SEQ ID No.3). As comparison the susceptible control ‘OP177’ is evaluated. The pepper line ‘E20B.30329’ and control ‘OP177’ are tested with a Pc isolate named Q108 as described in the above examples. The pepper line ‘E20B.30329’ and control ‘OP177’ are also tested with Xcv strains; race Xcv3 to Xcv6.

Resistance is scored on a range from 1 susceptible to 9 no symptoms.

In a resistant phenotype (8 or 9), the roots and shoots grow normally and no lesion is observed; in an intermediate resistance phenotype (4 to 7), the root growth is slowed, and although the plant is not collapsed, black lesions are visible; in a susceptible phenotype (1 to 3), root or shoot growth is stopped, and plant wilting and/or girdling is observed.

As shown in Table 11, ‘E20B.30329’ shows high resistance for both Pc and Xcv. This embodiment comprises both the Pc resistance trait described in preceding examples and a Xcv resistance trait.

TABLE 11
Results of Pc and Xcv resistance phenotyping.

| | |
|---|---|
| Resistance | Pathogen Tested |

| score | Pc | Xcv3 | Xcv4 | Xcv5 | Xcv6 |
|---|---|---|---|---|---|
| ‘E20B.30329’ | 7 | 9 | 9 | 9 | 9 |
| ‘OP177’ | 1 | 1 | 1 | 1 | 1 |

The plant according to present invention shows to be resistance to both Pc as well as Xcv, all Xcv 3-6 stains tested. The control plant which does not comprise any of the Pc and Xcv resistance genes (SAR8.2, Xcv) or Pc resistance locus (Pc 6.1) is fully susceptible to both Xcv and Pc. ‘E20B.30329’ scores 7 for Pc, because it is heterozygous for both the SAR8.2 resistance gene and Pc6.1 resistance locus. A score of 8 or 9 is expected for an embodiment homozygous for both resistance genes. Seeds of E20B.30329 are deposited at NCIMB Ltd, Ferguson Building, Craibstone Estate, Bucksburn, Aberdeen, AB21 9YA Scotland, on 9 Mar. 2023 under the number NCIMB 44129.

1. A plant of the Capsicum annuum (C. annuum) species that is resistant to Phytophthora capsici, wherein the plant comprises
a SAR8.2 resistance gene, that encodes for a SAR8.2 resistance protein, having at least 90% amino acid sequence identity with SEQ ID NO: 2,
a Pc6.1 resistance locus, wherein the Pc6.1 resistance locus comprises a first marker comprising a T at position 54 of SEQ ID xxx5 and/or a second marker comprising an A at position 101 of SEQ ID NO: 6, and
a Xcv resistance gene, that encodes for a resistance Xcv protein having at least 95% amino acid sequence identity with SEQ ID NO: 4.

2. The plant according to claim 1, wherein the SAR8.2 resistance gene comprises a coding sequence having at least 90% sequence identity with SEQ ID NO: 1.

3. The plant according to claim 1, wherein the Xcv resistance gene comprises a coding sequence having at least 90% sequence identity with SEQ ID NO: 3.

4. The plant according to claim 1, wherein the SAR8.2 resistance gene comprises at least one of the mutations selected from the group consisting of G48C, A179G and A14G as compared to a SAR8.2 gene encoding SEQ ID NO: 9, that does not provide resistance to Phytophthora capsica in a plant of the C. annuum species.

5. The plant according to any one of the claim 1, wherein the Pc6.1 resistance locus is flanked by SEQ ID NO: 7 and SEQ ID NO: 8.

6. The plant according to claim 1, wherein the plant is furthermore resistant to Xanthomonas campestris pv. vesicatoria (Xcv).

7. The plant according to claim 1, wherein the plant is resistant to one or more of Xanthomonas campestris pv. vesicatoria (Xcv) races 3, 4, 5 and/or 6.

8. The plant according to claim 1, wherein the SAR8.2 resistance gene is homozygous or heterozygous present in the genome of said plant, preferably homozygous, and/or,
wherein the Pc6.1 locus is homozygous or heterozygous present in the genome of said plant, preferably homozygous, and/or,
wherein the Xcv resistance gene is homozygous or heterozygous present in the genome of said plant, preferably homozygous.

9. (canceled)

10. (canceled)

11. The plant according to claim 1, wherein the plant is a sweet bell pepper.

12. The plant according to claim 1, wherein the SAR8.2 gene is obtainable from deposit accession number NCIMB 44129, and/or
wherein the Pc6.1 resistance locus is obtainable from deposit accession number NCIMB 44129, and/or
wherein the Xcv resistance gene is obtainable from deposit accession number NCIMB 44129.

13. (canceled)

14. (canceled)

15. A seed, tissue, plant cell or plant part of the plant of claim 1, wherein the seed, tissue, or plant part comprises the SAR8.2 resistance gene, Pc6.1 resistance locus and Xcv resistance gene.

16. A SAR8.2 resistance gene in combination with a Pc6.1 resistance locus and a Xcv resistance gene in a C. annuum plant for providing resistance to Phytophthora capsica, wherein
the SAR8.2 resistance gene comprises a coding sequence having at least 90% sequence identity with SEQ ID NO: 1,
the Pc6.1 resistance locus, wherein the Pc6.1 resistance locus is flanked by SEQ ID NO: 7 and SEQ ID NO: 8,
the Xcv resistance gene comprises a coding sequence having at least 90% sequence identity with SEQ ID NO: 3.

17. The SAR8.2 resistance gene in combination with the Pc6.1 resistance locus and the Xcv resistance gene in the C. annuum plant according to claim 16, wherein the SAR8.2, Pc6.1 and Xcv resistance genes are heterozygous or homozygous, preferably a combination thereof, more preferably all homozygous, present in said C. annuum plant.

18. The SAR8.2 resistance gene in combination with the Pc6.1 resistance locus and the Xcv resistance gene in the C. annuum plant according to claim 16, wherein said combination furthermore provides for resistance to Xanthomonas campestris pv. vesicatoria (Xcv).

19. A DNA construct comprising the SAR8.2 resistance gene in combination with the Pc6.1 resistance locus and/or the Xcv resistance gene according to claim 16, wherein one or more of said genes and/or said locus is operably linked to a promoter.

20. A method for providing a plant of the C. annuum species according to claim 1 wherein the method comprises the steps of;
a) selecting a pepper plant that is resistant to Phytophthora capsici, wherein said selection comprises establishing the presence of one or more resistance genes or locus selected from the group consisting of the SAR8.2 resistance gene, the Pc6.1 resistance locus and the Xcv resistance gene, wherein
the SAR8.2 resistance gene comprises a coding sequence having at least 90% sequence identity with SEQ ID NO: 1,
the Pc6.1 resistance locus comprises a first marker comprising a T at position 54 of SEQ ID NO: 5 and/or a second marker comprising an A at position 101 of SEQ ID NO: 6, and;
the Xcv resistance gene comprises a coding sequence having at least 90% sequence identity with SEQ ID NO: 3, and
b) transferring, for example by crossing, the identified one or more resistance genes of step a) into a C. annuum plant thereby conferring Phytophthora capsici resistance to said C. annuum plant, and
c) optionally, perform steps a) and b) until the obtained C. annuum comprises the SAR8.2 resistance gene, the Xcv resistance gene and the Pc6.1 resistance locus in its genome.

21. The method according to claim 20, wherein after step b) a first C. annuum plant is selected to be resistant to Phytophthora capsici and is crossed with a second C. annuum plant that is not resistant to Phytophthora capsici, and subsequently selecting a C. annuum plant that is resistant to Phytophthora capsici.

22. The method according to claim 20, wherein in step a) establishing the presence of the SAR8.2 resistance gene in C. annuum plant is performed by a marker comprising SEQ ID NO: 1, and/or
wherein the presence of the Pc6.1 resistance locus in C. annuum plant is established by a marker comprising a T at position 54 of SEQ ID NO: 5 and/or a marker comprising an A at position 101 of SEQ ID NO: 6 and/or wherein the resistance locus is flanked by a marker comprising SEQ ID NO: 7 and/or a marker comprising SEQ ID NO: 8, and/or
wherein the presence of the Xcv resistance gene in C. annuum plant is established by a marker comprising SEQ ID NO: 3.

23. (canceled)

24. (canceled)

25. The method according to claim 20, wherein the plant of the C. annuum species provided, is resistant to Phytophthora capsica and Xanthomonas campestris pv. Vesicatoria.

26. A method, comprising using a marker for establishing the presence of the SAR8.2 resistance gene in combination with the Pc6.1 resistance locus and the Xcv resistance gene according to claim 16 in a pepper plant,
wherein
for the SAR8.2 resistance gene the marker comprises SEQ ID NO: 1,
for the Pc6.1 resistance locus the marker is one or more selected from the group consisting of SEQ ID NO: 5, SEQ ID NO: 6, and SEQ ID NO: 7 and SEQ ID NO: 8, and
for the Xcv resistance gene the marker comprises SEQ ID NO: 3.
